(19) United States
(12) Patent Application Publication
(10) Pub. No.: US 2004/0225203 A1
(43) Pub. Date: Nov. 11, 2004

(54) Real-time and simultaneous monitoring of multiple parameters from multiple living beings

(76) Inventors: Mae C. Jemison, Houston, TX (US); Erick Kindred, Webster, TX (US); Raj Mandavilli, Los Gatos, CA (US)

Correspondence Address: Lekha Gopalakrishnan, JENKENS & GILCHRIST, A PROFESSIONAL CORPORATION, 1445 Ross Avenue, Suite 3200, Dallas, TX, 75202, US

(21) Appl. No.: 10/839,386

(22) Filed: May 5, 2004

Related U.S. Application Data
(60) Provisional application No. 60/468,308, filed on May 6, 2003.

(51) Int. Cl. A61B 5/00

(52) U.S. Cl. 600/300; 128/903

(57) ABSTRACT

The invention relates generally to monitoring physiologic and non-physiologic parameters associated with one or more living beings. These parameters can include physiologic and non-physiologic information regarding the living being. The parameters from all living beings are monitored in real-time at the same time, thus providing many advanced capabilities including, but not limited to, (i) comparing the performance of one living being to another; (ii) studying an living being's performance at various times of the day; (iii) comparing the physiology of one living being to another; (iv) comparing the physiology of a living being to a group of individuals; and (v) comparing the physiology of one group of living beings to another group of living beings.

### PRIOR RELATED APPLICATIONS

This application claims the benefit of U.S. Provisional Application No. 60/468,308, filed May 6, 2003.

### FEDERALLY SPONSORED RESEARCH STATEMENT

Not applicable.

### REFERENCE TO MICROFICHE APPENDIX

Not applicable.

### FIELD OF THE INVENTION

The invention relates generally to monitoring physiologic and non-physiologic parameters associated with one or more living beings. These parameters can include physiologic and non-physiologic information regarding the living being. The parameters from all living beings are monitored in real-time at the same time, thus providing many advanced capabilities including, but not limited to, (i) comparing the performance of one living being to another; (ii) studying an living being's performance at various times of the day; (iii) comparing the physiology of one living being to another; (iv) comparing the physiology of a living being to a group of individuals; and (v) comparing the physiology of one group of living beings to another group of living beings.

### BACKGROUND OF THE INVENTION

Currently available systems primarily monitor physiologic parameters of patients in hospitals and home. These systems are primarily used in the treatment of patients. In addition, the currently available systems do not monitor parameters that reflect the autonomic nervous system (ANS). A person's autonomic nervous system controls and regulates many of the physiologic housekeeping chores required for daily living, generally with minimal conscious awareness or interference. The ANS is responsible for one's automatic bodily functions, such as breathing, heartbeat, sweating, blood vessel dilation and contraction and glandular secretions—any of the myriad of responses our bodies automatically have to stimuli. The ANS responds to a wide range of externally and internally generated stimuli—heat and cold, exertion, fear, anger and elation, a box of chocolate candy, impending mortgage foreclosure or profuse bleeding. In a sense, measurements of the ANS are our best barometer to how we are “getting along” in our environment. The ANS has a critical role as a mediator between people's perception of the world around them and their bodies' immediate and long-term responses to that world.

ANS measurements provide crucial scientific information on and insight into our emotions, physical response to our environment and overall physiologic and neurological health. As such, ANS data can be beneficial in behavioral therapy, stress management, medical and psychiatric diagnosis, fitness assessment and sports and mental/psychological performance. The most valid and therapeutically helpful data is that which is gathered in the actual circumstances where and when maladies and challenges occur—at work, shopping, at the gym, in a business conference or at home. So minimizing measurement impact on daily lifestyles maximizes the benefit to the users.

U.S. Pat. No. 5,873,369 to Laniado et al. discloses a method to monitor the health condition of an individual and to detect potential dangers. Upon detecting danger, a warning signal is generated. This system does not have the capability to monitor multiple individuals. There is also no capability to monitor in real-time.

U.S. Pat. No. 6,035,230 to Kang et al. discloses a method for real-time biological signal monitoring. This system does not have the capability to monitor multiple individuals.

U.S. Pat. No. 6,246,992 to Brown discloses a method multiple patient monitoring system for proactive health management. This system does not have the capability to monitor in real-time and the patient need not be mobile.

U.S. Pat. No. 6,364,834 to Reuss et al. discloses a method and system for remotely monitoring multiple medical parameters. This system monitors parameters of a single individual. There is no real-time monitoring capability.

U.S. Pat. No. 5,907,291 to Chen et al. discloses a method for multi-patient monitoring. This system requires a bank of receivers to obtain the data from the telemetry devices. In other words, there is a receiver for each telemetry device. A unique frequency is necessary for each telemetry device. The system does not have the capability to send data over long distances (such as over the Internet). The telemetry device can only be used on patients in hospitals and it does not provide full ambulatory capabilities. The system does not have the capability of showing any combination of parameters on a single graph.

U.S. Pat. No. 6,616,606 to Petersen et al. discloses a method for patient monitoring system. This system does not have the capability to compare different parameters of a single individual, compare same parameter of multiple individuals, compare different parameters of multiple individuals, and perform statistical calculations. In addition, the system does not have the capability of showing any combination of parameters on a single graph.

There is therefore a need for a system that simultaneously measures one or more physiological and non-physiological parameters of one or more living beings in real-time and in a manner that is completely ambulatory, i.e., where the one or more individuals are free to move around at will without being tethered to the monitoring device(s). Embodiments of the invention disclosed and claimed herein provide many advanced capabilities such as (i) comparing the performance of one living being to another (ii) studying a living being's performance at various times of the day (iii) comparing the physiology of one living being to another (iv) comparing the physiology of a living being to a group of living beings (v) and comparing the physiology of one group of living beings to another group of living beings.

### SUMMARY OF THE INVENTION

An embodiment of the invention provides a system to monitor physiological and other parameters of more than one living being in real-time simultaneously. Physiologic parameters of the living being may include, but are not limited to, electrocardiogram measurements, respiration and body temperature. Non-physiologic parameters of the individual include motion, acceleration, and position. As used herein, the term “living being” is intended to encompass individuals in the human population, including adults and children, as well as non-humans such as members of the animal kingdom.

In certain embodiments of the invention, the living being is a human. In other embodiments of the invention, the living being may be a member of the animal kingdom.

An embodiment of the invention comprises at least two components. A first component is an ambulatory device worn by the living beings who are being monitored. This device measures, collects, and stores various physiologic and non- physiologic parameters with respect to the individual being monitored. The ambulatory device also sends information wirelessly to a second component of the invention, the monitoring station. The monitoring station receives information from these ambulatory devices wirelessly in real-time. This monitoring station can therefore display and analyze all the information in real-time or near real-time. This embodiment of the invention, therefore, displays the capability to monitor multiple parameters from multiple living beings in real-time in a simultaneous manner.

An embodiment of the invention provides a method for monitoring parameters of one or more living beings comprising, mounting one or more ambulatory devices on said one or more living beings, wherein said ambulatory devices do not restrict the movement of said living beings; collecting data pertaining to one or more parameters of said one or more living beings through the ambulatory devices; and monitoring the data collected through the ambulatory devices at a monitoring station.

A further embodiment of the invention recites a system for monitoring parameters of one or more living beings comprising, at least one ambulatory device configured to monitor one or more parameters of a living being, and at least one monitoring station, where the at least one monitoring station receives data from the at least one ambulatory device and records said data.

### BRIEF DESCRIPTION OF THE DRAWINGS

FIG. 1 shows a schematic of an ambulatory device.

FIG. 2 shows the placement of electrocardiogram electrodes.

FIG. 3 shows a schematic of a monitoring station.

FIG. 4 shows a typical configuration of a “multiple-parameter multiple- individual monitoring” system.

FIG. 5 illustrates the addition of a second monitoring station.

FIG. 6 illustrates the addition of monitoring stations over the Internet.

### DESCRIPTION OF EXEMPLARY EMBODIMENTS

An embodiment of the invention relates to a “multiple-parameter multiple-individual monitoring” system comprising at least two components—an ambulatory device and a monitoring station. As used herein, the term “ambulatory device” is intended to refer to a device which upon mounting on a living being, does not require the individual to be tethered to the system. The mounting of an ambulatory device on an individual does not restrict the living being's freedom of movement to any degree.

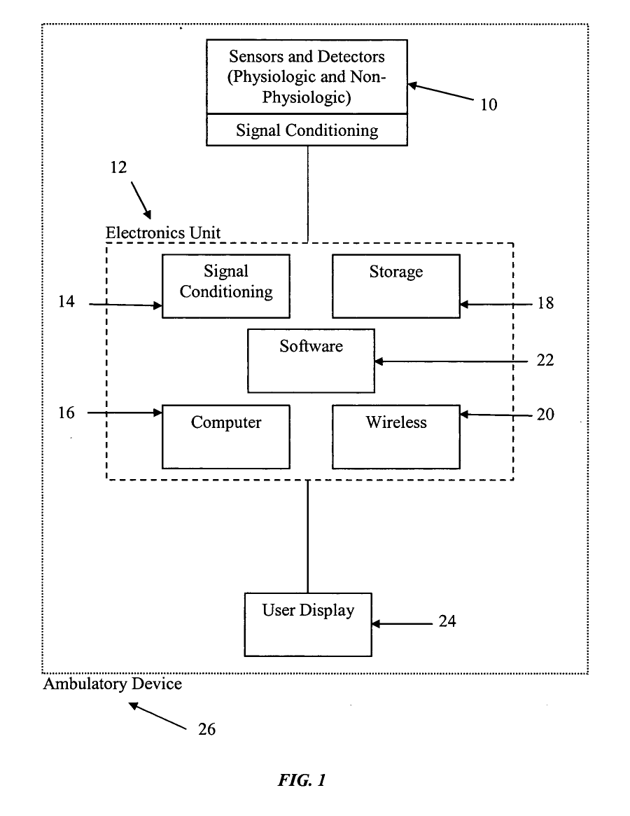

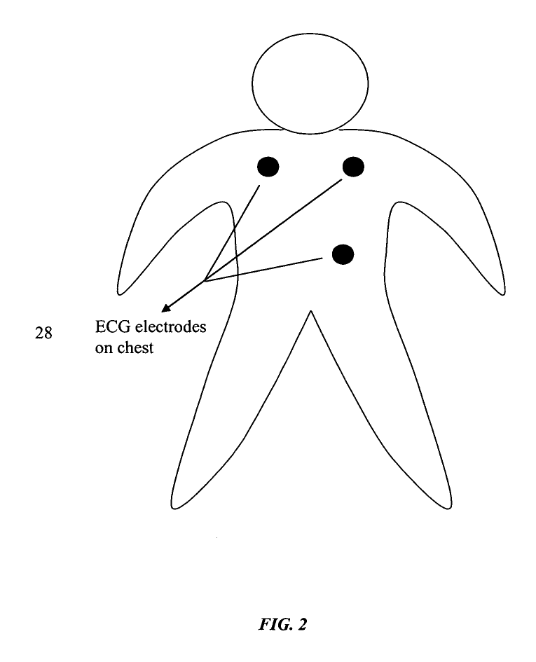

FIG. 1 shows a schematic of an ambulatory device used in an embodiment of the invention as used on an individual. The sensors and detectors 10 that measure physiologic and non-physiologic parameters are mounted/placed on the individual's body at the appropriate locations. For example, to measure an electrocardiogram (ECG), three electrodes 28 are placed on the chest as shown in FIG. 2 and the ECG sensors are attached to these electrodes. To measure acceleration/position, an accelerometer is placed on the chest or head. Some signal conditioning may be performed near the sensor/detector. Signal conditioning removes noise from the signals measured by the sensors/detectors. A garment may be used to hold the sensors/detectors in place on the individual. The garment also hides and guides the wires from these sensors/detectors.

Information from the sensors/detectors is sent to the electronics unit 12. The electronics unit is comprised of sub-components. The signal conditioning sub-component 14 helps to remove noise from the signals coming from the sensors/detectors 10. It also helps to amplify the signals. The computer sub-component 16 contains the processor, memory to perform the necessary computations. For example, the heart rate may be calculated from the electrocardiogram (ECG) signal. The storage sub-component 18 is used to store the information/data from the sensors/detectors and also to store data generated by other analyses. The wireless sub-component 20 sends (and receives) information and data to the monitoring station. The software sub-component 22 manages and coordinates all the above four sub-components—do the signal-conditioning, store data, do the wireless transmission and reception, and use the processor.

The user display 24 is used to display information to the individual. The display can be worn on the wrist, for example. Parameters such as heart rate, body temperature, blood pressure, and others could be displayed.

In the current embodiment, the ambulatory device 26 measures the following physiologic parameters: (i) Electrocardiogram (ECG) (ii) Respiration (iii) Volume of blood flow through the finger called Blood Volume Pulse (iv) Temperature of finger and (v) Skin Conductance Level at the wrist, a measure of sweating. These parameters reflect the ANS. Each measurement is a specific and sensitive indicator of the dynamic state of the ANS. Heart rate and R-R Interval are derived from the ECG. In addition, parameters such as Skin Conductance Level and Blood Volume Pulse can be measured at locations such as the ankle.

Additional parameters can be measured in alternate embodiments of the invention including, but not limited to, physiologic parameters such as (i) Oxygen saturation at the finger, a measure of oxygen in the blood (ii) Core body temperature (iii) Blood pressure (iv) Blood sugar (v) Electroencephalogram (EEG), brain signals. Metabolism and amount of calories burnt over a certain period can be calculated. Non- physiologic parameters include position, motion, and acceleration.

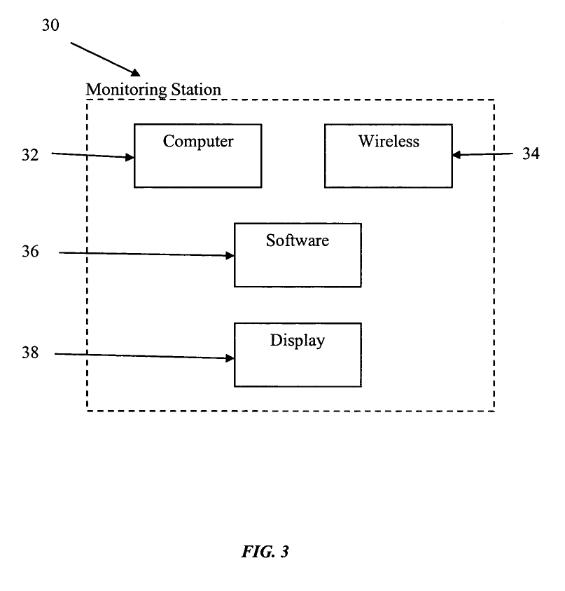

A second component of a “multiple-parameter multiple-individual monitoring” system is a monitoring station, as indicated in the schematic diagram shown in FIG. 3. The monitoring station 30 comprises four sub-components. The wireless sub- component 34 receives (and sends) data or information from the ambulatory device(s) worn by individual(s). The computer 32 could be a Windows operating system based laptop or desktop computer. Other operating systems such as Linux and other computers such as Mac could also be used. The software 36 performs all analyses and derivations. For example, Heart Rate Variability, a measure of heart rate variations, is derived from the Electrocardiogram (ECG). Various statistical calculations can be performed on the data received from the ambulatory devices. For example, the average rate of the entire group or a subset could be calculated. The display 38 is used to show the data in the desired format. For example, two-dimensional graphs can be used to show the change of respiration rate with respect to time. Three-dimensional graphs may be used to plot the frequency spectrum of Heart Rate Variability.

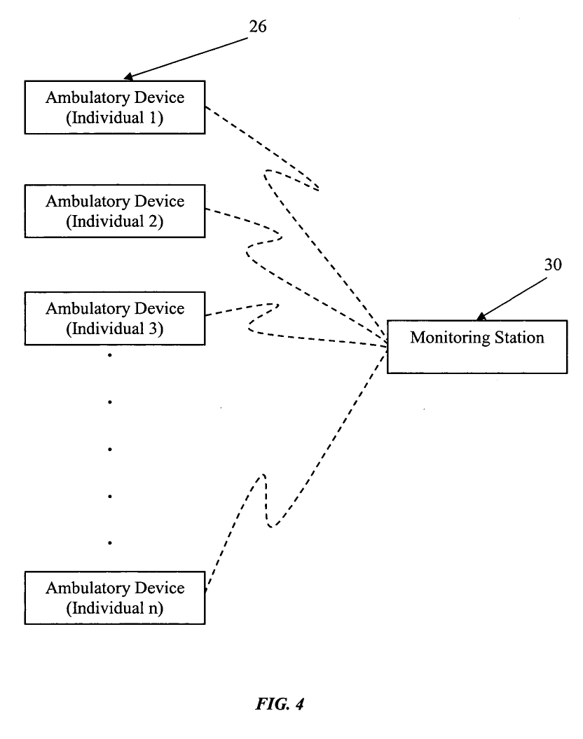

A typical configuration of a “multiple-parameter multiple-individual monitoring” system is illustrated in FIG. 4. Each individual being monitored puts on an ambulatory device. The ambulatory device will enable the individual to be mobile, since the device is small and all sensors and sensor leads are all hidden. Once the ambulatory device 26 is turned on, the device starts measuring the physiologic and non-physiologic data from the individual. The ambulatory device's functions/capabilities are outlined below:

i. The physiologic and non-physiologic data can be stored on the ambulatory device.

ii. The physiologic and non-physiologic data can be transmitted to the monitoring station.

iii. The ambulatory device can also receive information/instructions from the monitoring station.

iv. The ambulatory device displays appropriate information to the individual.

v. The sensors and detectors are held in place on the individual's body by the ambulatory device.

vi. The wires or leads from the sensor and detectors are hidden by the ambulatory device. Therefore, the device does not constrict the individual's movements.

vii. The ambulatory device removes any noise from the measurements and performs signal conditioning.

viii. The ambulatory device can calculate some parameters from the measurements, and if necessary display them to the individual.

A monitoring station 30 in FIG. 4 receives the data from the many ambulatory devices (individuals). The monitoring station's functions/capabilities are outlined below:

i. Multiple parameters of one individual can be monitored in real-time at the same time. For example, one can view graphs in real-time of multiple parameters of one individual.

ii. A single parameter of many individuals can be monitored in real-time at the same time. For instance, one can view graphs in real-time of a single parameter of many individuals.

iii. Multiple parameters of many individuals can be monitored in real-time at the same time. For example, one can view graphs in real-time of multiple parameters of many individuals.

iv. Statistical and mathematical calculations can be performed in real-time (or near real-time) on parameters from a single individual or parameters from a few individuals or parameters from the entire group of individuals.

v. Ability to find outliers.

vi. Ability to compare one parameter to another parameter of a single individual in real-time.

vii. Ability to compare the same parameter of two or more individuals in real-time.

viii. Ability to compare one parameter of one individual to a different parameter of another individual.

ix. Data is received wirelessly from the ambulatory devices.

x. All data can be stored on the monitoring station.

xi. Instructions/Data can also be sent back to the ambulatory device(s) and displayed to the individual, if necessary.

xii. The entire saved “monitoring session” can be played back.

xiii. The entire saved “monitoring session” can be loaded at the same time to view all of the data simultaneously. Therefore, one can zoom in to a particular region to study it more.

From the description above, a number of advantages of the “multiple- parameter multiple-individual monitoring” become evident:

1. The system facilitates measurement of more than one physiologic and non- physiologic parameter.

2. The system facilitates measurement of various parameters of multiple individuals.

3. The system gives the capability to compare data of one individual to that of another.

4. The system gives the ability to use one monitoring station for many individuals rather than use a monitoring station for each individual.

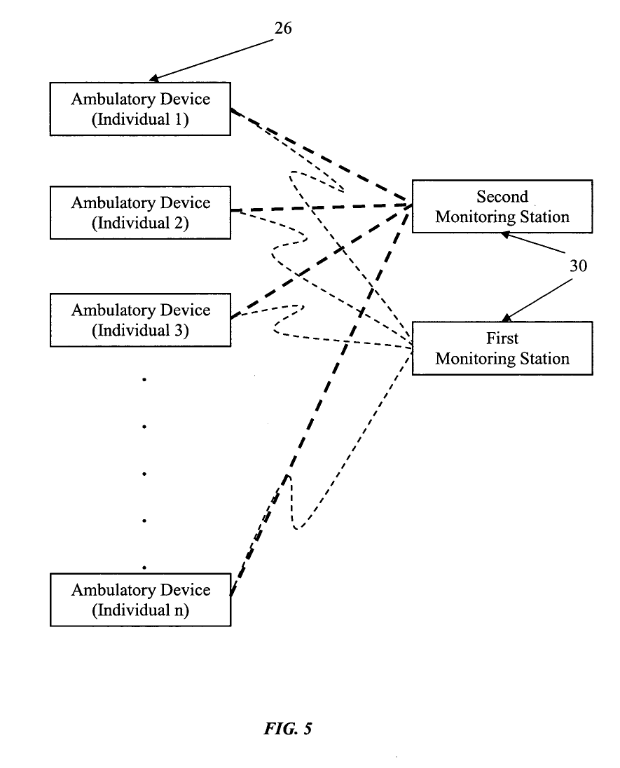

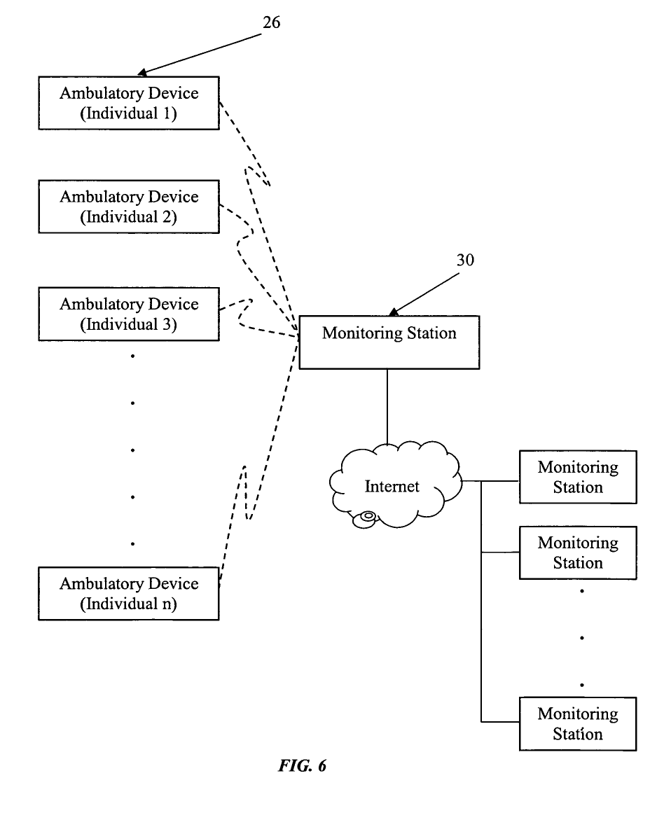

If necessary, additional monitoring stations can be utilized in the system as illustrated in FIG. 5 and FIG. 6. In FIG. 5, a second monitoring station is added. In FIG. 6, monitoring stations are added in locations that are outside the wireless range. These monitoring stations can be connected via the Internet. Therefore, a person located in California can monitor individuals in New York, for example.

The “multiple-parameter multiple-individual monitoring” system has a number of applications. In an embodiment of the invention, the system can be used to monitor firefighters while they are fighting fires. An individual from a safe distance can monitor the physiology, location, and movement of the firefighters. Frequently, most firefighters fail to recognize that they are exhausted and therefore lose consciousness due to exhaustion. About 40-50% of firefighter deaths occur due to stress or overexertion. By monitoring the firefighters, the individual can recognize those firefighters near exhaustion and ask them to pull out prior to losing consciousness.

In an alternate embodiment of the invention, professional military personnel such as soldiers in the military can be monitored while in training and during war. A soldier's performance can be monitored under stressful situations and the soldier can be trained to deal with these stresses. If a soldier is injured, the medic will get advance information on the physiological status of this soldier. This may help the medic and/or doctor plan the treatment in advance.

In an embodiment of the invention, the system can be used to monitor the performance of professional athletes in football, basketball, soccer, athletics etc. An athlete's performance can be monitored during different times of the day and peak performance times can be evaluated.

In further embodiments of the invention, team dynamics can be evaluated by studying the interactions of one athlete to others. For example, the performance of NASCAR drivers can be monitored. The use of this system is not restricted to individuals in professional sports. For instance, individuals in gyms can also be monitored.

In an embodiment of the invention, the system can be used in couples therapy by studying the interaction of couples during therapy and at other places/situations, group therapy by studying the interaction of teams or groups during therapy and at other places/situations, and anger management by monitoring the individual for anger during therapy and during situations where the individual experiences anger.

Pharmaceutical companies can use an embodiment of the invention to monitor the behavior of subjects during clinical trials of their drugs. This should help the pharmaceutical companies observe the effects of their drugs in real-time and thus possibly reduce time to bring a drug to market.

An embodiment of the invention can also be used in healthcare. For example, the physiology of senior citizens, human adults greater than 65 years of age, can be monitored while they are at home. The physiology of a patient who underwent surgery can be monitored after they are sent home. An embodiment of the invention can also be used to screen for Diabetic Autonomic Neuropathy, a common ailment suffered by diabetics.

An embodiment of the invention can be used for the monitoring of persons suffering from a sleep apnea disorder. Typically, an individual suffering from sleep apnea symptoms is observed while sleeping in the hospital or clinic. The state-of-the-art equipment have numerous wires going from the subject's body to the equipment, therefore, restricting the movements during sleep. The constrained movements in combination with factors such as sleeping in a hospital contribute to the subject/patient not getting a normal sleep pattern. This might affect the observation or study adversely. An embodiment of the invention may be used to overcome certain of the hurdles posed by the state-of-the-art equipment, thereby allowing the subject/patient to sleep under normal conditions at home.

Various researchers (clinical, physiological, psycho-physiological, ANS etc.) can use an embodiment of the invention to monitor the behavior of subjects for their research or studies. The subjects may be observed at home, at work, in the lab, or any other appropriate location.

A further embodiment of the invention may be used to monitor the physiology of animals. For example, the system can be used to monitor the physiology of a racehorse. Hence, the horse trainer can study the performance of the horse. This will enable the trainer and jockey to be able to judge when to “push” the horse to a faster pace. Human-animal interactions can also be monitored with this system. Using the example of the racehorse, a trainer will be able to judge whether a racehorse performs better with one jockey versus another.

1. A method for monitoring parameters of one or more living beings comprising:
mounting one or more ambulatory devices on said one or more living beings, wherein said ambulatory devices do not restrict the movement of said living beings;
collecting data pertaining to one or more parameters of said one or more living beings through the ambulatory devices in a simultaneous manner; and monitoring the data collected through the ambulatory devices at a monitoring station in a simultaneous manner.

2. The method of claim 1, wherein said parameters are either physiologic or non- physiologic parameters.

3. The method of claim 1, wherein said parameters are monitored in real time.

4. The method of claim 2, wherein said parameters are selected from the group consisting of electrocardiogram, respiration, blood volume pulse, finger temperature, degree of sweating, level of blood oxygen, core body temperature, blood pressure, blood sugar and electroencephalogram.

5. A method of comparing one or more parameters of a group of living beings comprising:
mounting one or more ambulatory devices on said group of living beings, wherein said ambulatory devices do not restrict the movement of said group of living beings;
collecting data pertaining to one or more parameters of said group of living beings through the ambulatory devices in a simultaneous manner; and
monitoring the data collected through the ambulatory devices at a monitoring station in a simultaneous manner.

6. The method of claim 5, wherein said parameters are either physiologic or non- physiologic parameters.

7. The method of claim 5, wherein said parameters are monitored in real time.

8. The method of claim 6, wherein said parameters are selected from the group consisting of electrocardiogram, respiration, blood volume pulse, finger temperature, degree of sweating, level of blood oxygen, core body temperature, blood pressure, blood sugar and electroencephalogram.

9. A method of comparing one or more parameters of a living being comprising:
mounting one or more ambulatory devices on said living being, wherein said ambulatory devices do not restrict the movement of said living being;
collecting data pertaining to one or more parameters of said living being through the ambulatory devices in a simultaneous manner; and
monitoring the data collected through the ambulatory devices at a monitoring station in a simultaneous manner.

10. The method of claim 9, wherein said parameters are either physiologic or non- physiologic parameters.

11. The method of claim 9, wherein said parameters are monitored in real time.

12. The method of claim 10, wherein said parameters are selected from the group consisting of electrocardiogram, respiration, blood volume pulse, finger temperature, degree of sweating, level of blood oxygen, core body temperature, blood pressure, blood sugar and electroencephalogram.

13. The method of claim 1, wherein the living being is a human.

14. The method of claim 1, wherein the living being is an animal.

15. The method of claim 13 wherein, the human is an athlete.

16. The method of claim 13 wherein, the human is a military professional.

17. The method of claim 13 wherein, the human is a fire fighter.

18. The method of claim 13 wherein, the human is a senior citizen.

19. The method of claim 13 wherein, the human is a subject in a clinical trial.

20. The method of claim 13 wherein, the human is a subject in a research study.

21. The method of claim 13 wherein, the human suffers from sleep apnea.

22. The method of claim 14 wherein, the animal is a horse.

23. A system for monitoring parameters of one or more living beings comprising:
at least one ambulatory device configured to monitor one or more parameters of an individual; and
at least one monitoring station;
where the at least one monitoring station receives data from the at least one ambulatory device and records said data.

24. The system of claim 23, wherein said parameters are either physiologic or non- physiologic parameters.

25. The system of claim 23, wherein said parameters are monitored in real time.

26. The system of claim 23, wherein said parameters are selected from the group consisting of electrocardiogram, respiration, blood volume pulse, finger temperature, degree of sweating, level of blood oxygen, core body temperature, blood pressure, blood sugar and electroencephalogram.
